(19) United States
(12) Patent Application Publication
(10) Pub. No.: US 2015/0190649 A1
(43) Pub. Date: Jul. 9, 2015

(54) EMBEDDED PHOTONIC SYSTEMS AND METHODS FOR IRRADIATION OF MEDIUM WITH SAME

(71) Applicant: The General Hospital Corporation, Boston, MA (US)

(72) Inventors: Jeffrey A. Gelfand, Cambridge, MA (US); Timothy Brauns, Roslindale, MA (US)

(21) Appl. No.: 14/410,743

(22) PCT Filed: Jun. 27, 2013
(86) PCT No.: PCT/US2013/048065

§ 371 Date: Dec. 23, 2014

Related U.S. Application Data
(60) Provisional application No. 61/666,099, filed on Jun. 29, 2012.

(51) Int. Cl. A61N 5/06; F21V 8/00; G02B 6/10

(52) U.S. Cl. CPC A61N 5/0601; G02B 6/10; G02B 6/0008; A61N 2005/0667; A61N 2005/0655

(57) ABSTRACT

System and method for irradiating a region of interest of a medical device, implanted in a biological tissue, and the surrounding tissue. The system includes a translucent wall having an optically diffusing component and, optionally, an associated source of light embedded therein. The optical diffuser is optionally configured to outcouple light with a uniform spatial distribution and may include a network of biocompatible waveguides. The system may include a catheter with a distal end with an embedded diffuser that outcouples light delivered from the external source through a spiral of optical fiber, buried in the wall of the catheter. The system may include a biocompatible layer permanently embedded into the biological tissue and containing quantum dots that are activated wirelessly, through the layer, to irradiate the surrounding tissue from inside. Such a layer does not need to be removed from the tissue after irradiation of the tissue has been accomplished.

### CROSS-REFERENCE TO RELATED APPLICATIONS

The present application claims the benefit of and priority from the U.S. Provisional Patent Application No. 61/666,099 filed on Jun. 29, 2012 and titled “Embedded Photonic Systems and Methods for Irradiation of Medium”. The disclosure of the above-mentioned provisional patent application is incorporated herein by reference in its entirety.

### TECHNICAL FIELD

The present invention relates generally to systems and methods of delivery of electromagnetic radiation inside biological tissue and, more particularly, to activating and/or assisting photodynamic therapeutic processes with the use of such delivery.

### BACKGROUND ART

The delivery of electromagnetic radiation (EMR) and, in particular, light for light-tissue interaction has been described in related art. Optically-controlled methods such as photodynamic therapy (PDT), photo-thermal therapy, low-level laser therapy, and light-activated drug release, to name just few, continue to emerge. Various applications of PDT are being explored, from the repair of injured skin and subcutaneous biological structures through the use of non-ablative collagen remodeling (so-called NCR technique) to the treatment of tumors through the use of low-energy light to generated reactive oxygen species in photosensitized target cells , to light-based disinfection of catheter tubes.

Some PDT procedures require light delivery deep into the tissue. The related art discussed the use of fiber-optic-based catheters or lens-based endoscopes for light delivery into a body. While efficient delivery of light to the tissue for the purposes antibacterial PDT is very important in clinical applications, systems and methods addressing such delivery are subject to at least one of numerous shortcomings. For example, they may require the use of a photosensitizer, which introduces an inevitable delay between the application of the PS and the beginning of the treatment itself. The use of photosensitizers in the disinfection of cathetersis also limited to intra-luminal disinfection, as the PS can only be introduced into the inside of the tube. Currently, the use of photodynamic therapy to treat bacterial and fungal colonization of catheter tube lumens requires a procedure performed by skilled clinical staff, thereby adding expense and complexity to the treatment procedure, including the required ordering and scheduling of the treatment procedure.

There remains a need to improving this kind of approach with a method that facilitates light delivery into the biological tissue for the purpose of bacterial disinfection that does not require the participation of skilled clinical personnel and ensures that optimal disinfection covers all areas of the medical implant as well as the surrounding tissue.

### SUMMARY OF THE INVENTION

Embodiments of the present invention provide an article of manufacture comprising a body defined by a wall and having a region of interest (ROI), and an element embedded into the wall. In a specific implementation, the article of manufacture may include at least a portion of a system, for delivery of light into the biological tissue for the purposes of PDT, that is to be permanently embedded into the tissue. The wall is generally configured to deliver excitation power from outside of the body through the wall to the ROI, and the element is configured to form a spatial distribution of electromagnetic radiation in response to the delivered excitation power.

In one implementation, the wall is tubular and, optionally, includes an optical waveguide passing through and/or along the wall. Such waveguide includes, in one embodiment, a fiber-optic (FO) component defining a three-dimensional (3D) spiral. The ROI may include a distal end of the body in which an optical diffuser, adapted to outcoupled light delivered to the distal end through the wall of the body, is implanted. In this case, the excitation power includes light power. Optionally, the optical diffuser is structured to ensure that the spatial distribution of the outcoupled light is substantially uniform. The element embedded in the wall may be spatially separated from ambient medium and, in a specific implementation, includes at least one quantum dot adapted to emit the EMR at a corresponding predetermined wavelength (for example, an IR wavelength or a wavelength corresponding to visible light), or a QD light0emitting diode layer.

When the body of the article of manufacture includes proximal and distal ends and the ROI includes the distal end of the body, the article may additional include an electrical connector extending between the proximal and distal ends. Alternatively or in addition, the element embedded into the wall includes a transformer configured to transform the excitation power received wirelessly by the transformer, to at least one of electrical power and light.

The article of manufacture may additionally contain a source of the excitation power in operable communication with the element embedded into the wall. In a specific embodiment, such source is adapted to vary at least one characteristic of the excitation power.

Embodiments of the invention additionally provide a method for irradiation of a medium with an optical diffuser having a body defined by a wall and an optical component embedded in the wall. Generally, the medium to be irradiated surrounds the optical diffuser, which, in a specific case, be permanently embedded into the medium. An embodiment of the method includes (i) receiving, at the optical component and through the wall, excitation energy from a source of energy located outside of the medium; and (ii) forming, with the optical diffuser, a substantially spatially-uniform distribution of electromagnetic radiation (EMR) in response to the received excitation energy such that the EMR impinges onto the medium. The method may include generating the EMR at the optical component and, in a specific case, generating light at a quantum dot (QD) embedded in the wall of the optical diffuser. The optical diffuser, in turn, can be implanted in a biological tissue that is a target of irradiation with the EMR. The optical diffuser may include a QD light-emitting diode layer. The excitation energy can optionally be received by the QD, wirelessly. The formation of a substantially spatially-uniform distribution of EMR may involve the outcoupling of light delivered to the optical component along a lightguide that is embedded in the wall and that forms a three-dimensional (3D) spiral. Generally, the excitation energy received by the optical component can be received via an electrical connector, a lightguide, or wirelessly.

### BRIEF DESCRIPTION OF THE DRAWINGS

The invention will be more fully understood by referring to the following Detailed Description of Specific Embodiments in conjunction with the Drawings, of which:

FIG. 1 is an article of manufacture according to an embodiment of the invention.

FIG. 2 shows a related article of manufacture.

FIGS. 3 and 4A are diagrams each of which illustrates an alternative embodiment of the invention containing a sources of light embedded in a wall of the embodiment.

FIGS. 4B and 4C are diagrams showing alternative embodiments of wireless activation of a source of light of FIGS. 3 and 4A.

FIG. 5 is a diagraph showing an embodiment having a tubular wall.

FIG. 6 is a diagram illustrating an optical channel of delivery of light associated with the embodiment of FIG. 5.

FIGS. 7A, 7B are diagrams illustrating fabrication of quantum-dot light-emitting layers for use with an embodiment of the invention.

### DETAILED DESCRIPTION

In accordance with embodiments of the present invention, a method and apparatus are disclosed for irradiating the areas of ambient medium (such as biological tissue, for example) that are susceptible to growth of a biofilm or infection, resulting from intrusion of a foreign object (for example, due to implantation of a medical device), with EMR (and, in particular IR light). The radiation towards the ambient medium is generally activated with excitation energy from a source that is external to the ambient medium.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and/or in reference to a figure, is intended to provide a complete description of all features of the invention.

In addition, in drawings, with reference to which the following disclosure may describe features of the invention, like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and not all, features of the invention. A given drawing and an associated portion of the disclosure containing a description referencing such drawing do not, generally, contain all elements of a particular view or all features that can be presented is this view in order to simplify the given drawing and the discussion, and to direct the discussion to particular elements that are featured in this drawing.

A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described features, structures, or characteristics of the invention may be combined in any suitable manner in one or more embodiments.

Moreover, if the schematic flow chart diagram is included, it is generally set forth as a logical flow-chart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Without loss of generality, the order in which processing steps or particular methods occur may or may not strictly adhere to the order of the corresponding steps shown.

The invention as recited in the appended claims is intended to be assessed in light of the disclosure as a whole.

Medical devices that are implanted or inserted into a biological tissue may expose the tissue to colonization by pathogens such as bacteria and may alter the tissue environment by wounding, lacerating, or ulcerating the surrounding tissue. Such collateral damage extends healing times, produces areas of chronic inflammation, causes pain and discomfort to the patients, and may lead to the premature failure of the implanted medical device.

The antibacterial effect of visible light irradiation used in combination with photosensitizers and by itself is quite advantageous. Visible light, for example, phototoxically affects various microorganisms and cultures, including those associated with periodontal diseases and the use of indwelling catheters, especially in long-term central venous catheters. In addition to reducing detrimental effects and recovery complications (such as blood stream infections, for example), resulting from inflammatory processes caused by various bio-contaminations, the disinfection of various elements cooperated with a patient's body reduce an economic burden on the health care system. Delivery of UVC light intra-luminally to the catheter hubs and tubes, for example, with the use of an UVC LED light source attached to a standard Luer catheter was reported to substantially reduce the contamination of the tube with microorganisms. The use of the UV therapy for direct photo-deactivation of the bacteria has several limitations, not the least of which is that the antibacterial effects produced by the UV light are associated with DNA damage and represents safety risks for humans. In comparison, the irradiation of the catheters with blue light relies on endogenous photosensitizing agents (such as porphyrin) within the bacteria, and the use of this specific chromophore inevitably limits the efficiency of irradiation for diverse strains of bacteria. In addition, the use of blue light is not known to be particularly effective in treatment of fungi. In contrast, phototreatment of bacteria with specific wavelengths of IR radiation that are not dependent on organism-specific chromophores and are unlikely to cause DNA damage may have a potential to preserve mammalian cells while still producing antibacterial effect at clinically-realistic dosages of irradiation. This alternative, direct photo-deactivation approach, sometimes called the “light-oxygen effect,” manifesting itself in generation of singlet oxygen without the use of photosensitive dyes. Other IR wavelengths of light have been linked to an ability to promote wound healing and tissue growth. Embodiments of the invention employ systems adapted to be implanted into a biological medium and containing embedded photonic components structured to leverage the use of the light-oxygen effect and/or photohealing can be employed in clinical situations calling for both direct and indirect generation of light without a direct connection to a EMR power generator (such as a light source), which remains distanced from the medium to which the EMR is applied.

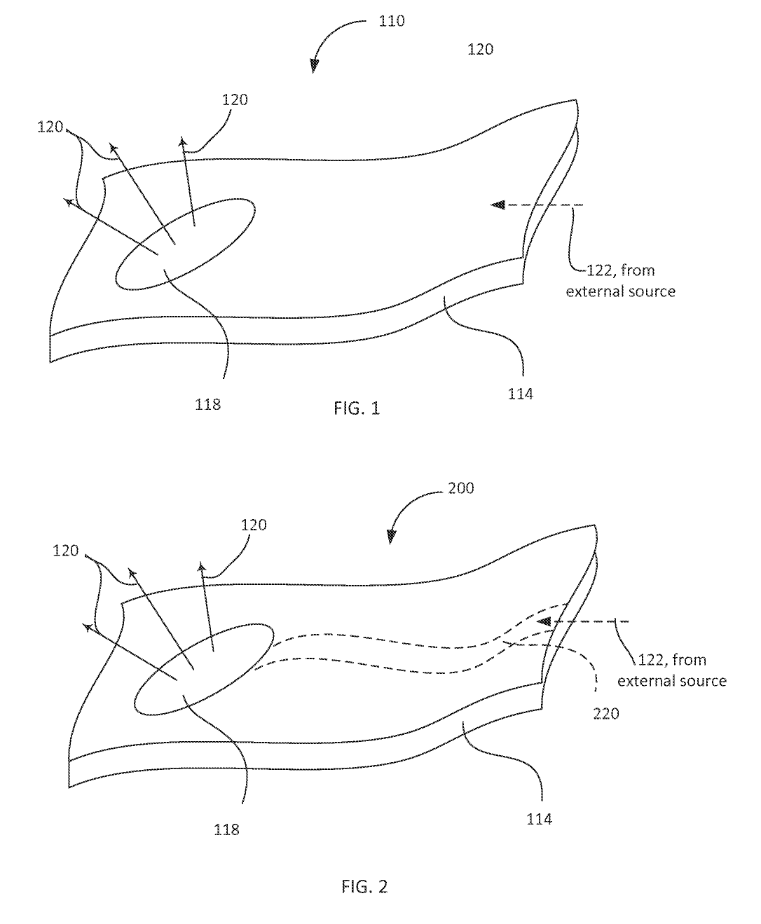

The idea of the invention stems from the realization that delivery of disinfecting radiation and/or radiation causing tissue-healing therapeutic effects to the region of interest (ROI) of the tissue with the use of an optical component semi-permanently (or permanently) embedded in the tissue and activated without direct contact with the layer substantially increases the efficiency of PDT, reduces traumatic effects associated with the need to periodically remove the component, and reduces the costs and complexity of PDT. FIG. 1 of the invention schematically illustrates an implementation of this idea. An optical component 110 is defined by a wall 114 that contains a ROI 118. The ROI 118 has EMR-diffusing properties (for example, the ROI 118 may incorporate an optically-diffusing component embedded into the wall 114) such that the EMR, delivered to or generated within the bounds of the ROI 118, is outcoupled from the wall 114 in a preferably spatially-uniform fashion to the surrounding medium as shown with arrows 120. Examples of such optically-diffusing component are provided by components made of materials such as silica, quartz, acrylic or perfluorinated polymers such as polyperfluorobutenylvinylether. In one implementation, the wall 114 is structured to operate as a slab guide of the EMR, delivered to the wall 114 from an external source of EMR towards the ROI 118, as shown with an arrow 122. In a related implementation 200 of FIG. 2, the wall 114 incorporates a channel waveguide or lightpipe element 200. In a related case, such lightpipe is embedded within the wall and structured to deliver the EMR (for example, light or IR radiation, or UV radiation) from an external source (not shown) toward the ROI 118 through and /or within the wall 114. The waveguiding structure 220 can be formed, for example, by photodefinition in a portion of the wall 114, or by lamination of the lightpipe within the wall 114. The ROI 118 of the material layer having the wall 114 may, in one embodiment, include a waveguide mesh or a network of waveguiding components such as those discussed, for example, in a commonly assigned U.S. Provisional Patent Application No. 61/561,191 filed on Nov. 17, 2011 and titled “Systems and Methods for Facilitating Optical Processes In Biological Tissue”. The disclosure of this provisional patent application is incorporated herein by reference in its entirety.

The embedding medium includes, in one case, a biological tissue in which an embodiment of the invention is implanted or embedded, such as soft tissue or a joint.

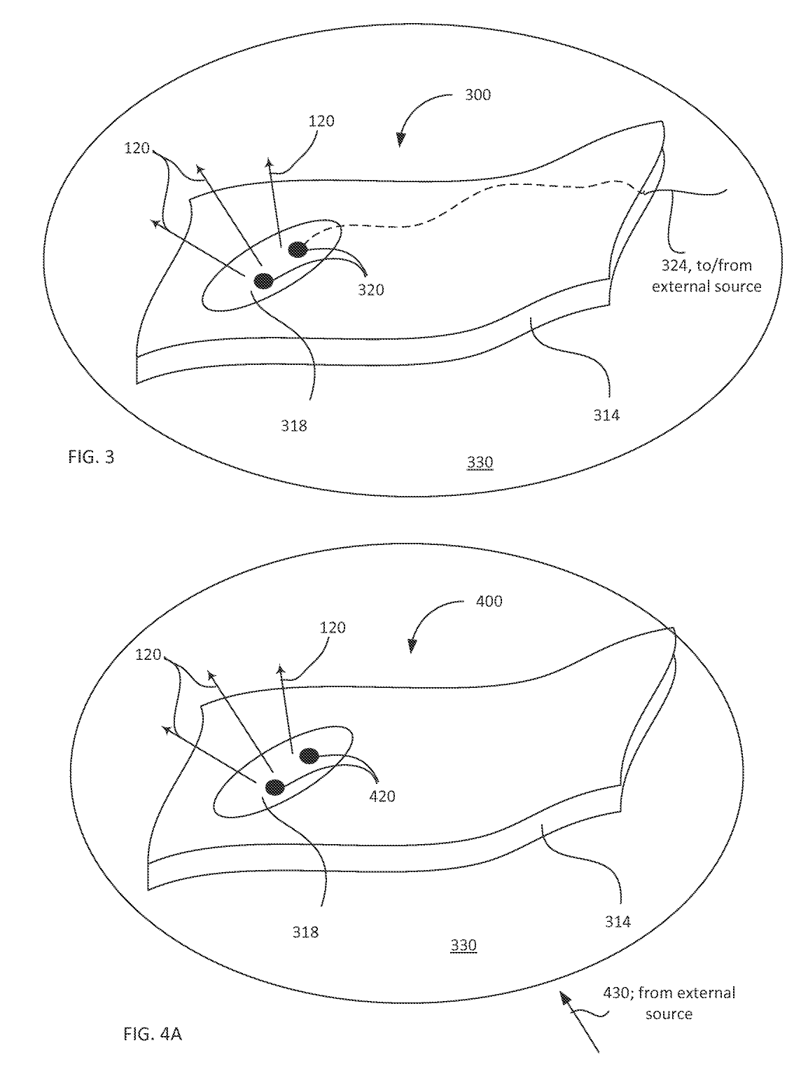

FIG. 3 illustrates a specifically-implemented device including an optical layer 300 defined by a wall 314. An ROI 318 of the wall possesses optically-diffusing characteristics and contains at least one embedded source of light 320 that may optionally be supplemented with a rechargeable microbattery and/or electronic circuitry (not shown) also directly embedded in the wall 314 or, alternatively, placed in operational proximity to the source of light 320 in the ambient medium 330 that surrounds the layer 300. In one implementation the source of light 320 includes a quantum dot light emitter. The use of LED, OLEDs, or other optical sources is also within the scope of the invention. The excitation for the source of light 320 is provided from an external source of excitation power via an excitation channel, which is defined by the nature of the excitation power. As shown in FIG. 3, for example, an electrically-conductive conduit 324 such as a thin wire made of material having electrical conductivity and size sufficient to conduct direct current of relatively low amperage (for example, at about or below 0.5 A), can be operably connected at one end to the source of light 320 and at the other end to the source of current and/or voltage (not shown). Such source of current and/or voltage is optionally controlled with a pre-programmed processor. An example of the conduit 324 is provided by a wire made of copper, silver or gold, to name just a few, and of typically less than AWG 30 gauge or diameter of about 0.25 mm When excited, the source of light 320 emits light within an associated bandwidth, and the optically-diffusive ROI 318 transmits such emitted light outwardly from the ROI 318 into the ambient medium 330. In a specific implementation, the layer structure 300 includes a QD light-emitting diode layer (QD LED layered structure). In one non-limiting example, the QD LED layered structure includes polymer nanocomposite thin film(s) with a chosen material composition (which may contain one or more of lead sulphide, lead selenide, and indium arsenide) disposed in association with such nanocomposite film(s) with the use of one or more of spin-coating, dip-coating, inkjet printing, phase separation, and contact printing. The chosen material composition may be disposed on or in the polymer nanocomposite film(s) The nanocomposite film(s) is carried, in turn, by a layer of dielectric material. The QD LED layered structure also includes an electrical contact (made of gold, for example) electrically connected to the chosen material composition. The chosen material composition is appropriately tuned to facilitate, in operation of the device, emission of EMR at specific wavelengths such as, for example, 1270 nm. The overall average thickness of a specific QD LED layered structure is on the order of one micron, but can be varied depending on the operational characteristics and targets.

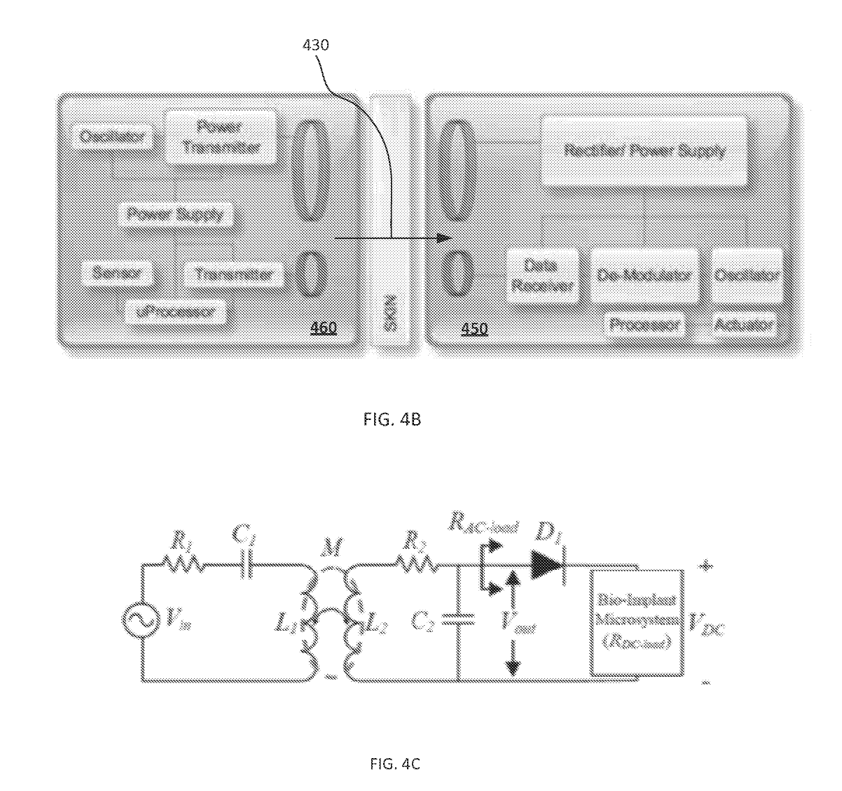

In comparison with the embodiment 300 of FIG. 3, the embodiment 400 of FIG. 4A is configured to embed or laminate at least one source of light 320 in the wall 314 such as to ensure wireless triggering or activation of the operation of the source of light 320 with an energy source that is external not only to the wall 314 but also to the medium 330 in which the wall 314 is housed. Such activation and delivery of the excitation power from the external energy source to the source of light 320 is provided through the wall 314 (as shown with an arrow 430—from below the layer 314).

In further reference to FIG. 4A, when the wireless activation of the source of light 320 is used, it is implemented with at least one of electromagnetic radiation, radiofrequency, microwave and ultrasound. Such wireless activation employs, for example, contactless recharging of a microbattery associated with the source of light 320. Alternatively or in addition, electrical current driving the source of light 320 may be directly generated in an embedded circuitry (not shown) via, for example, inductive coupling between operably connected coil systems. A simplified example of such coupling system, used to activate subcutaneously disposed circuitry 450 with the use of a circuitry portion 460 that is external to the biological tissue, is shown in FIG. 4B. Discussion of wireless energy conversion can be found, for example, in Laskovski A. N., Dissanayake T., and Yuce M. R., Wireless power technologies for biomedical implants. (Komorowska M. A., Olsztynska-Janus S., Eds., Biomedical Engineering, Vukovar, Croatia: InTech, 2009: 119-132). FIG. 4C illustrates a schematic diagram of electrical circuitry for wireless activation of an embodiment of the invention with the use of EMR at a radiofrequency. Alternatively, the activation of an implanted embodiment of the invention can be achieved with ultrasound, for example by analogy with powering of a piezoelectric middle ear device (see, for example, http://emedicine.medscape.com/article/1995195-overview). In either of these implementations employing wireless activation of an embedded device of the invention with the use of an external power source, auxiliary energy storage devices, such as capacitors or batteries, can be used to boost the current generated to drive the embedded device.

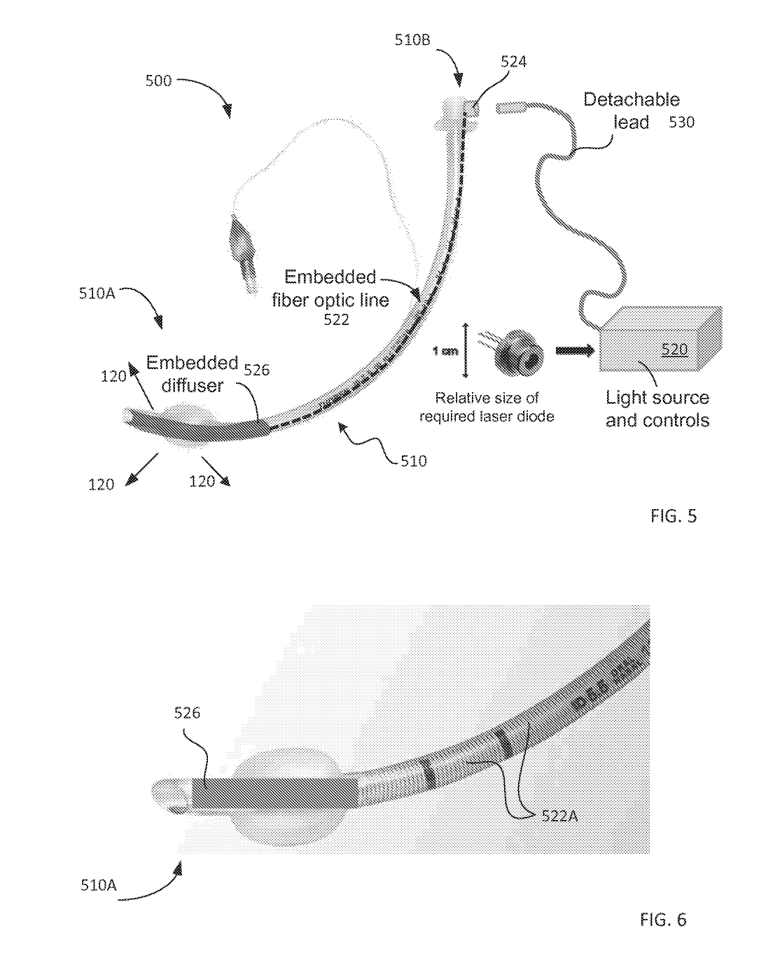

FIG. 5 illustrates a related embodiment 500 that utilizes a device of the invention having a tubular wall. The embodiment 500 includes a catheter or tube or stent or shunt 510 having distal and proximal ends 510A, 510B and a source of excitation power 520 that is external to and, optionally, removably connectable to the proximal end 510b of the tube 510 via a detachable lead 530. The component 510 can be used as part of an endotracheal tube, nasal tube, gastric tube, urinary catheter, central line catheter, PICC line, other CVP lines, portacath, Hickman catheter, nephrostomy stent, biliary stent, CNS shunt, and dialysis shunts (either regular or peritoneal). The catheter or tube 510 contains an optical fiber line (or, alternatively, a channel waveguide line) 522 embedded in the tube 510, a port 524 of which is adjacent to and/or cooperated with the proximal end 510b. The catheter or tube 510 also contains an optically-diffusing element 526 implanted or embedded in or at the distal end 510A. In one embodiment, the diffuser 526 and/or the tube 510 is adapted to form a substantially spatially-uniform distribution of light output.

Achieving spatial uniformity of light-output from the diffuser 526 and/or the tube 510 is advantageous in that such distribution allows the user of the system to optimize (for example, decrease) the overall light-output power required for disinfection of the surrounding tissue. (An example of such optimization is provided by minimization of fluence for a given exposure dose.) The spatial uniformity of light-output from the diffuser 526 is promoted by formatting the diffuser 526 out of an (optionally flexible) waveguide-network such as a waveguide-mesh or fiber-optic-woven cloth of sorts, embedded in a region of the tissue where the irradiation of tissue is required. A waveguides or fiber in such a waveguide-network or “cloth” is appropriately structured to “leak” light at a chosen rate along the length of the waveguide or fiber. For example, the combination of the core and cladding parameters of the optical fiber can be adapted to achieve form side-emitting segments, along the length of the fiber. Alternatively or in addition, the diffuser 526 may include a hollow cylinder composed of light-scattering material. Similarly, the spatial uniformity of light-output distribution along the tube 510 can be facilitated by the use, in association with the tube 510, of an optical fiber or cable that is likewise adapted to “leak” light along propagation over the fiber or cable. Alternatively or in addition, a relatively uniform distribution of light-output from an optical fiber can be achieved by configuring the fiber in a helical orientation in the region where light-emission is desired. In examples described above, the optical fiber associated with the tube 510 and delivering light to the diffuser 526 is operably interfaced with the diffuser 526. The light-diffusing components can be incorporated in a medical implant device during the manufacture of such a device using one of available techniques (such as, for example, that currently employed to produce endotracheal tubes that contain helical metal reinforcements, for example. Medical implantable/embeddable devices that can benefit from the light-outcoupling system and/or method for disinfecting of the ambient tissue of the present invention include a pacemaker and auto-implantable defibrillator, such as those equipped with their own independent power sources.

The source 520 (such as, for example, a light source and electronic circuitry adapted to effectuate the operation of the light source) transmits the excitation power (in one embodiment—the EMR and, in particular, light), through the lead 530 towards the port 524, when connected, and further down the fiberoptic line 522 towards the diffuser 526. The diffuser 526 outcouples light 120 towards the surrounding medium in which the distal end 510a is inserted or implanted. While the optical fiber line 522 is generally passing through and along the wall of the tuber 510, in a specific embodiment shown in FIG. 6 such line 522A is structured as a three-dimensional spiral extending, in the wall of the tube 510, between the proximal and distal ends 510A, 510B and establishing optical communication between the port 524 and the diffuser 526. The spiral-shaped fiber optic line 522 is, optionally, built in the wall of the tube 510 during the process of tube manufacturing. Alternatively, the fiber optic line 522 is wound or coiled around an existing tube and overcoated (to laminate the line 522) with a plastic overlayer the properties of which (including mechanical properties and biocompatibility) are similar to those of the material of the tube 510.

It is appreciated that a source of energy or power used to activate the operation of an embodiment of the invention is generally adapted to change or modify at least one characteristic of the excitation energy that it produces. In reference to FIG. 5, for example, the electronic circuitry associated with the source 520 is configured to define at least one of the frequency, amplitude, and phase of excitation light delivered to the embedded diffuser 526.

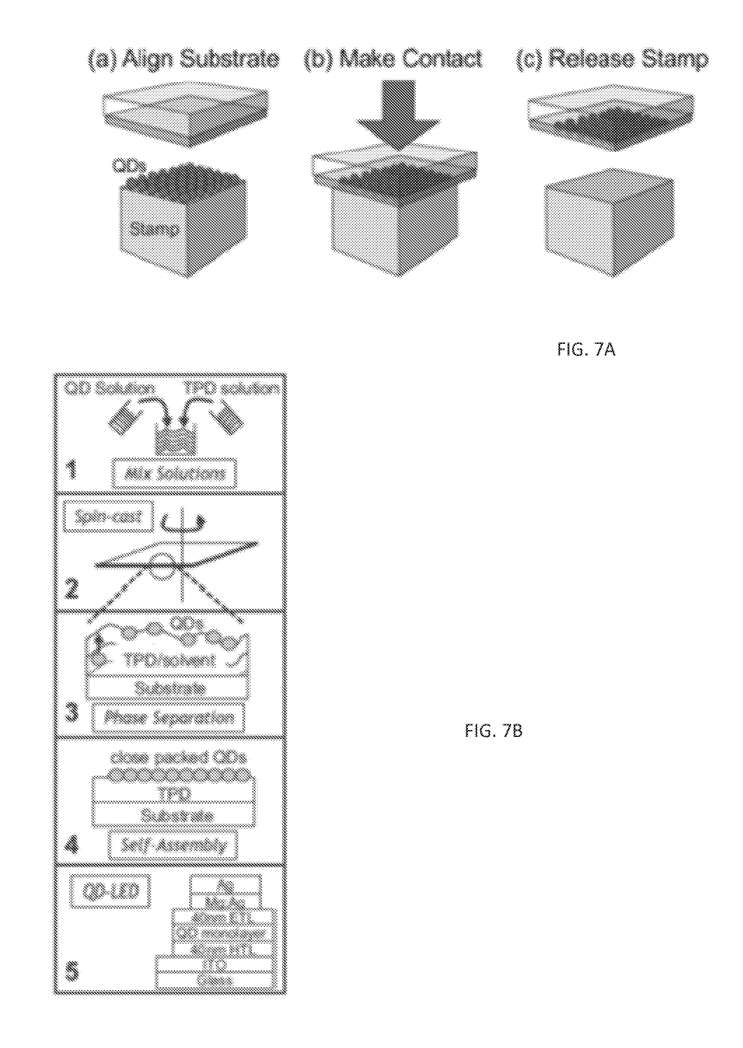

As mentioned above, an embodiment of the invention may employ a QD or a layer of QDs as a source of light to be implanted in a tissue of choice. Several method of manufacture of such layers of QDs are being currently developed. One method, referred to as phase separation, is a fabrication technique suitable for forming large area of ordered monolayers of QDs. A single layer of QDs is formed by spin-casting a mixed solution of aromatic organic materials (transport layers) and aliphatically-capped QDs. This process simultaneously yields QD monolayers self-assembled into hexagonally close-packed arrays and places this monolayer on top of a co-deposited contact. During solvent drying, the QDs phase separate from the organic under-layer material and rise towards the surface of the film. Another method, referred to as contact printing, is a solvent-free method (i.e., during the contact printing process the device structure is not exposed to solvents). Since charge transport layers in QD-LED structure are solvent-sensitive organic thin films, avoiding solvent during process is one of the major benefits of contact printing method. This method can produce patterned electroluminescent structures with 1000 ppi (pixels-per-inch) print resolution. Diagrams schematically illustrating the use of the phase separation and contact printing method of fabrication of the QD LED layers are shown, respectively, in FIGS. 7A and 7B.

Applications of embodiments of the invention include, for example, medical applications targeting disinfection of medical devices implanted into the biological tissue. For example, an endotracheal tube with embedded photonic component such as the embodiment 500 of FIG. 5 can be used to prevent pneumonia associated with insertion of catheters into the patient for the purposes of ventilation. Ventilator-assisted pneumonia (VAP) is caused, primarily, by biofilm formation on endotracheal tubes (ETTs), afflicts about 10% to 20% of all such patients; such infections extend ICU stays by an average of about 6 days and represent about 3.4B USD in additional medical expenditures per year in the U.S. Current approaches to reducing VAP include the use of silver ions microdispersed in association with an outer and/or inner surfaces of the lumen of the employed catheter. Such a solution is costly (about and exceeding $100 per tube) and its use can be undermined by the development of bacterial resistance to the silver.

Generally, and in accordance with specific embodiments described in reference to FIGS. 1 through 6, embodiments of the invention include an embedded optically-diffusing component (i.e., a diffuser, which is optionally equipped with a light-emitting element) in association with the implanted medical device (for example, below the surface of such medical device or in a wall of such medical device). The optically-diffusing component is configured to outcouple and diffuse infrared light in a manner that results in a uniform photon irradiance at the surface of the medical device. The diffuser may be embedded in the medical device such as to be out of contact with the surrounding biological tissue during the operation of the device. The material used for integration of the diffuser (with an optional light-emitting element) and the medical device may be transparent or translucent. The diffusers may be composed of networks of optical fibers designed for uniform release of light over the length of the fiber, optical fiber-based textiles that provide uniform light irradiance, or light-emitting films such as QD LED films or layers adapted to convert electrical impulses into light at specific wavelengths. Light-emitting films or layers may include multiple types of quantum dots capable of emitting different wavelengths of light for the purpose of microbial disinfection or tissue wound healing and cytoprotection. The diffuser is connected to a source of power (a control device) that provides either a specific wavelength of light or electrical power that drives the release of photons in the diffuse towards the surrounding medium and/or device. The power source is equipped with a control system providing for variable operation characteristics of the embodiment. For example, the control device may be adapted to emit light (at a level of about 1 W or lower) at wavelengths chosen to provide bacterial disinfection as well as tissue healing. The connection between the control device and diffuser may employ an optical channel (such as a waveguide or optical fiber), an electrical connector (such as a wire), a printed circuit, or a wireless connection established with the use of ultrasound or microwaves, for example.

The material of the medical device hosting the diffuser may utilize specific materials chosen to optimize the conversion of locally present oxygen molecules into singlet oxygen species when specific infrared wavelengths of light are used. This feature of an embodiment of the invention allows its use in absence of use of photosensitizing compounds introduced onto the device or the surrounding tissue. A device of the invention may be additionally coated to absorb light at specific wavelengths in order to increase the efficiency of conversion of photonic energy and localize such conversion at the surface of the medical device. Combinations of photoabsorbing coatings and aromatic polymers disposed on a surface of the implanted medical device may be additionally used to localize the release of singlet oxygen as a means of disinfecting surfaces of the medical device.

In a specific embodiment, medical devices are hollow tubes placed in the body such as endotracheal tubes, catheters, stents and shunts. The external portion of the tubular wall is structured to have a receptacle to accept either photonic or electrical input intended to be channeled to the diffuser embedded in the tube.

In one implementation, a method of the invention is implemented by having an operator select a wavelength, power setting and exposure duration from the control device that is suitable for the type of treatment desired. The types of envisioned treatments include microbial disinfection of the medical device implanted into the biological tissue (such as a knee joint or soft tissue, for example) or wound healing/cytoprotection of the healthy tissue surrounding the implanted medical device. As an example only, light outcoupled by the diffuser towards a surface of the medical device and/or the tissue includes lights in a spectral region of about 10 nm FWHM centered at about 760 nm, 870 nm, 900 nm, 930 nm, 1064 nm and 1270 nm (for disinfection purposes, for example) and light at about 760 nm, 808-810 nm, 850 nm, 880-890 nm, 1064 nm and 1270 nm for wound healing and cytoprotection.

At least some elements of a device of the invention can be controlled, in operation, with a processor governed by instructions stored in a tangible, non-transitory storage medium. Such storage medium may include random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

Modifications to, and variations of, the illustrated embodiments may be made without departing from the inventive concepts disclosed herein. Furthermore, disclosed aspects, or portions of these aspects, may be combined in ways not listed above. Accordingly, the invention should not be viewed as being limited to the disclosed embodiment(s).

What is claimed is:

1. An article of manufacture comprising
a body defined by a wall and having a region of interest (ROI) therein, said wall configured to deliver excitation power from outside of the body through the wall to the ROI; and
an element embedded into the wall at the ROI, said element configured to form a spatial distribution of electro-magnetic radiation (EMR) in response to excitation power delivered from outside of the body through the wall to the ROI.

2. An article according to claim 1, wherein said wall is tubular.

3. An article according to claim 2, wherein said tubular wall is configured as an optical waveguide.

4. An article according to claim 2, further comprising an optical waveguide disposed in the tubular wall.

5. An article according to claim 4, wherein said optical waveguide includes a fiber-optic (FO) component defining a three-dimensional (3D) spiral.

6. An article according to claim 1, wherein said ROI includes a distal end of the body, said excitation power includes light power, and said element includes an optical diffuser structured to outcouple light delivered to the distal end through said wall to define a substantially spatially-uniform distribution of irradiance of the outcoupled light.

7. An article according to claim 6, wherein said optical diffuser includes one or more of a cylindrical element and a waveguide network.

8. An article according to claim 7, wherein said waveguide network includes a fiber-optic mesh.

9. An article according to claim 1, wherein said element is spatially separated from ambient medium outside the body.

10. An article according to claim 1, wherein said element includes a first quantum dot adapted to emit EMR at a first wavelength.

11. An article according to claim 10, wherein said EMR at a first wavelength includes one or more of visually-perceivable EMR and infra-red (IR) light.

12. An article according to claim 10, wherein said element includes a second quantum dot adapted to emit EMR at a second wavelength.

13. An article according to claim 10, wherein said body includes proximal and distal ends and said ROI includes the distal end, and further comprising an electrical connector extending between the proximal and distal ends.

14. An article according to claim 10, wherein said element includes a transformer configured to transform said excitation power, received wirelessly by said element, to at least one of electrical power and light.

15. An article according to claim 1, wherein said element includes a quantum dot (QD) light-emitting diode layer.

16. An article according to claim 1, further comprising a source of said excitation power in operable communication with said element, the source being adapted to vary at least one characteristic of said excitation power.

17. An article according to claim 1, wherein said element is adapted to form a spatial distribution of EMR configured to disinfect at least a portion of a medium surrounding said element.

18. A method of irradiation of a medium with an optical diffuser having a body defined by a wall and an optical component embedded in the wall, the medium surrounding said optical diffuser, the method comprising:
receiving, at the optical component and through the wall, excitation energy from a source of energy located outside of the medium; and
forming, with said optical diffuser, a substantially spatially-uniform distribution of electromagnetic radiation (EMR) in response to the received excitation energy, said EMR impinging onto the medium.

19. A method according to claim 18, wherein said forming includes generating said EMR at the optical component.

20. A method according to claim 18, wherein said forming includes generating light at a quantum dot (QD) embedded in the wall of said optical diffuser.

21. A method according to claim 20, wherein said receiving includes wirelessly receiving excitation energy by said QD.

22. A method according to claim 18, wherein said receiving includes receiving excitation energy at the optical component embedded in a biological tissue.

23. A method according to claim 22, wherein said forming includes forming a substantially spatially-uniform distribution of light delivered to the optical component along a lightguide embedded in said wall, said lightguide defining a three-dimensional (3D) spiral.

24. A method according to claim 23, wherein said receiving includes receiving excitation energy delivered by at least one of an electrical connector, a lightguide, and wireless communication.
